(19) United States
(12) Patent Application Publication
(10) Pub. No.: US 2024/0391857 A1
(43) Pub. Date: Nov. 28, 2024

(54) ALCOHOL MIXTURES INCLUDING LINEAR TRIDECANOLS

(71) Applicant: Dow Global Technologies LLC, Midland, MI (US)

(72) Inventors: Michael A. Brammer, Lake Jackson, TX (US); Wanglin Yu, Pearland, TX (US)

(21) Appl. No.: 18/713,938

(22) PCT Filed: Dec. 1, 2022
(86) PCT No.: PCT/US2022/051497

§ 371 Date: May 28, 2024

Related U.S. Application Data
(60) Provisional application No. 63/286,129, filed on Dec. 6, 2021.

(51) Int. Cl. C07C 29/141; C07C 41/01

(52) U.S. Cl. CPC C07C 29/141; C07C 41/01

(57) ABSTRACT

An alcohol mixture includes greater than 90 wt % of a linear tridecanol based on the total weight of the alcohol mixture.

### BACKGROUND

### Field of the Disclosure

The present disclosure is directed to alcohol mixtures and more specifically to alcohol mixtures including linear tridecanols.

### INTRODUCTION

Alcohols having carbon atoms from C₈ to C₂₀ are known to be useful as precursors to produce surfactants. The alcohols are functionalized to include a hydrophilic moiety to form the surfactants. The hydrophilic moiety may be added to the alcohol though processes such as alkoxylation, glycosidation, sulfation, phosphation. The properties and application functions of the resulting surfactant materials highly depend on the chain length of alcohol precursors. A C₁₃ alcohol or an alcohol mixture centered at about C₁₃ is particularly useful for making surfactants that can find use in broad applications, including for example laundry and dish washing. The carbon atoms of the alcohol may be in a linear or branched structure. The branching structure of carbon atoms of the alcohol plays a critical role in determining the physical and application properties of a surfactant as well as the biodegradability of the surfactant. For example, hydrophobe branching dramatically affects foaming and performance in dishwashing applications as branching increases. Hydrophobe branching also significantly affects the wetting and gelation behavior of surfactants.

The most commonly used C₁₃ alcohol products in the industry are made through oligomerization of propylene or butene followed by hydroformylation. The C₁₃ alcohols made from these synthetic routes are highly branched. While branching can benefit fast wetting, solubility, and less foam-persistence of surfactants, it can also adversely affect biodegradability and cleaning efficiency. Process efforts have been made to reduce the branching in the oligomerization of C₃ or C₄ olefins for producing C₁₃ alcohols with lower degrees of branching. Another approach to produce C₁₃ alcohols with less degree of branching is to hydroformylate a C₁₂ linear alpha-olefin to aldehyde followed by hydrogenation, also known as the oxo process.

High linearity alcohols for use in surfactants has been attempted before. For example, rhodium catalyzed hydroformylation of higher alkenes has been able to produce alcohol linearities of between 80% to 90%, but linearities above 90% have yet been unachievable. See section 8.4.3 of Rhodium catalyzed hydroformylation (Vol. 22). Regardless, linearities above 90% for tridecanols have been avoided. For example, U.S. Pat. No. 9,828,565 (“the '565 patent”) provides a composition comprising a mixture of tridecanols wherein at least about 60 wt % of the mixture is linear tridecanol and at least about 10 wt % of the mixture is branched tridecanols. The '565 patent explains that “[t]he higher the branching content, the lower the pour point, affording ease and economy in processing” and that “[w]e find that alcohols having 10-40% branching provide an optimal tradeoff between low temperature solubility and soil removal for many surfactant derivatives.” The '565 patent also explains that the branched and linear tridecanols are separated using crystallization.

In view of the foregoing, it would be surprising to have an alcohol mixture that comprises greater than 90 wt % linear tridecanols and was useful in the formation of surfactants.

### SUMMARY OF THE DISCLOSURE

The inventors of the present application have discovered an alcohol mixture that comprises greater than 90 wt % linear tridecanols and is useful in the formation of surfactants. Such a surfactant is believed to have enhanced biodegradability due to the linearity of the alcohol used as an initiator. The surfactant is believed to offer sufficient cleaning performance to be used in a variety of applications. Also surprisingly discovered is that the oxo process can produce alcohol mixtures having greater than 90 wt % linear tridecanols without the need for separation processes to removed branched tridecanols.

The present disclosure is particularly useful for the formation of industrial materials.

According to a first feature of the present disclosure, an alcohol mixture comprises greater than 90 wt % of a linear tridecanol based on the total weight of the alcohol mixture.

According to a second feature of the present disclosure, 92 wt % or greater of the alcohol mixture is linear tridecanol based on the total weight of the alcohol mixture.

According to a third feature of the present disclosure, 94 wt % or greater of the alcohol mixture is linear tridecanol based on the total weight of the alcohol mixture.

According to a fourth feature of the present disclosure, 7 wt % or less of the alcohol mixture is branched tridecanol based on the total weight of the alcohol mixture.

According to a fifth feature of the present disclosure, a formulation, comprises 0.1 wt % to 99 wt % of the alcohol mixture of claim 1.

According to a sixth feature of the present disclosure, a method of making an alcohol mixture, comprises the steps of contacting a C₁₂ linear olefin with carbon monoxide, hydrogen and a catalyst composition to produce an aldehyde mixture; and hydrogenating the aldehyde mixture to produce an alcohol mixture comprising greater than 90 wt % of linear tridecanol based on the total weight of the alcohol mixture.

According to a seventh feature of the present disclosure, the method of making an alcohol mixture further comprises the step of distilling the alcohol mixture.

According to an eighth feature of the present disclosure a method of making a surfactant material comprising the step of performing an alkoxylation of the alcohol mixture.

### DETAILED DESCRIPTION

As used herein, the term “and/or,” when used in a list of two or more items, means that any one of the listed items can be employed by itself, or any combination of two or more of the listed items can be employed. For example, if a composition is described as containing components A, B, and/or C, the composition can contain A alone; B alone; C alone; A and B in combination; A and C in combination; B and C in combination; or A, B, and C in combination.

All ranges include endpoints unless otherwise stated.

As used herein, the term weight percent (“wt %”) designates the percentage by weight a constituent is of a total weight of a composition unless otherwise specified.

As used herein, Chemical Abstract Services registration numbers (“CAS #”) refer to the unique numeric identifier as most recently assigned as of the priority date of this document to a chemical compound by the Chemical Abstracts Service.

### Alcohol Mixture

The present disclosure is directed to an alcohol mixture. The alcohol mixture comprises a linear tridecanol. Linear tridecanols correspond to Structure (I):

H—CH₂—CH₂—CH₂—CH₂—CH₂—CH₂—CH₂—CH₂—CH₂—CH₂—CH₂—CH₂—CH₂—OH Structure (I)

The alcohol mixture comprises greater than 90 wt % of linear tridecanol based on the total weight of the alcohol mixture. For example, the alcohol mixture may comprise 90.1 wt % or greater, or 90.5 wt % or greater, or 91.0 wt % or greater, or 91.5 wt % or greater, or 92.0 wt % or greater, or 92.5 wt % or greater, or 93.0 wt % or greater, or 93.5 wt % or greater, or 94.0 wt % or greater, or 94.5 wt % or greater, or 95.0 wt % or greater, or 95.5 wt % or greater, while at the same time, 96.0 wt % or less, or 95.5 wt % or less, or 95.0 wt % or less, or 94.5 wt % or less, or 94.0 wt % or less, or 93.5 wt % or less, or 93.0 wt % or less, or 92.5 wt % or less, or 92.0 wt % or less, or 91.5 wt % or less, or 91.0 wt % or less, or 90.5 wt % or less of linear tridecanol based on the total weight of the total weight of the alcohol mixture.

The alcohol mixture may comprise branched tridecanols. Branched tridecanols may have Structure (II):

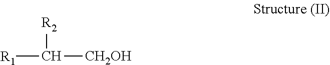

where R₁ and R₂ are linear alkyl chains containing a total of 11 carbon atoms in the two alkyl chains. It will be understood that although the branch position of Structure (II) is depicted at the C₂ location, other points of branching may be present in the branched tridecanol. The alcohol mixture may comprise 7 wt % or less of branched tridecanol. For example, the alcohol mixture may comprise 0.0 wt % or greater, or 0.1 wt % or greater, or 0.5 wt % or greater, or 1.0 wt % or greater, or 1.5 wt % or greater, or 2.0 wt % or greater, or 2.5 wt % or greater, or 3.0 wt % or greater, or 3.5 wt % or greater, or 4.0 wt % or greater, or 4.5 wt % or greater, or 5.5 wt % or greater, or 6.0 wt % or greater, or 6.5 wt % or greater, while at the same time, 7.0 wt % or less, or 6.5 wt % or less, or 6.0 wt % or less, or 5.5 wt % or less, or 5.0 wt % or less, or 4.5 wt % or less, or 4.0 wt % or less, or 3.5 wt % or less, or 3.0 wt % or less, or 2.5 wt % or less, or 2.0 wt % or less, or 1.5 wt % or less, or 1.0 wt % or less, or 0.5 wt % or less of branched tridecanol based on the total weight of the total weight of the alcohol mixture.

### Formulation

The alcohol mixture may be utilized in the formation of one or more formulations. For example, the formulation may be a plasticizer, a lubricant, a cleaning composition, a home care composition, a cosmetics composition, an industrial composition, a pharmaceutical, a personal care product or other material. The formulation comprises 0.1 wt % to 99 wt % of the alcohol mixture based on the total weight of the formulation. For example, the formulation may comprise 0.1 wt % or greater, or 1 wt % or greater, or 5 wt % or greater, or 10 wt % or greater, or 20 wt % or greater, or 30 wt % or greater, or 40 wt % or greater, or 50 wt % or greater, or 60 wt % or greater, or 70 wt % or greater, or 80 wt % or greater, or 90 wt % or greater, while at the same time, 99 wt % or less, or 90 wt % or less, or 80 wt % or less, or 70 wt % or less, or 60 wt % or less, or 50 wt % or less, or 40 wt % or less, or 30 wt % or less, or 20 wt % or less, or 10 wt % or less, or 1 wt % or less of the alcohol mixture based on the total weight of the formulation.

### Method of Making the Alcohol Mixture

The present disclosure is also directed to a method of making the alcohol mixture. The method of making the alcohol mixture comprises steps of (1) contacting a C₁₂ linear olefin with carbon monoxide, hydrogen and a catalyst composition to produce an aldehyde mixture followed by a second step of (2) hydrogenating the aldehyde mixture to produce an alcohol mixture comprising greater than 90 wt % of linear tridecanol based on the total weight of the alcohol mixture. Contacting olefins, hydrogen and carbon monoxide to produce aldehydes is generally known as hydroformylation. The reaction conditions and process characteristics are well known in the art. For example, the World Intellectual Property Organization publication number 2019231613A1 provides suitable reaction and process conditions for carrying out the hydroformylation of the present invention. The C₁₂ linear olefin can be terminally or internally unsaturated.

The hydrogen and carbon monoxide used in the method may be obtained from any suitable source, including petroleum cracking and refinery operations. The hydrogen and carbon monoxide used in the method may be pre-combined in the form of syngas. Syngas (from synthesis gas) is the name given to a gas mixture that contains varying amounts of CO and H₂. Production methods are well known. Hydrogen and CO typically are the main components of syngas, but syngas may contain CO₂ and inert gases such as N₂ and Ar. The molar ratio of H₂ to CO varies greatly but generally ranges from 1:100 to 100:1 and preferably between 1:10 and 10:1. Syngas is commercially available and is often used as a fuel source or as an intermediate for the production of other chemicals. The most preferred H₂:CO molar ratio for chemical production is between 3:1 and 1:3 and usually is targeted to be between about 1:2 and 2:1 for most hydroformylation applications.

A solvent may be employed in the hydroformylation process. Any suitable solvent that does not unduly interfere with the hydroformylation process can be used. By way of illustration, suitable solvents for rhodium catalyzed hydroformylation processes include those disclosed, for example, in U.S. Pat. Nos. 3,527,809; 4,148,830.

The catalyst composition used in the method comprises (a) a transition metal; (b) a monophosphine; and (c) a tetraphosphine having Structure (III):

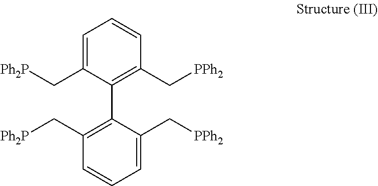

wherein P of Structure (III) represents a phosphorus atom and Ph₂ of Structure (III) represents two phenyl moieties such that each PPh₂ comprises a diphenylphosphino moiety.

The transition metal can include Group 8, 9 and 10 metals selected from rhodium (Rh), cobalt (Co), iridium (Ir), ruthenium (Ru), iron (Fe), nickel (Ni), palladium (Pd), platinum (Pt), osmium (Os) and mixtures thereof. In rhodium examples, the transition metal of the catalyst composition may be provided in the form of a stable crystalline solid such as rhodium hydridocarbonyl-tris(triphenyl phosphine), or rhodium dicarbonyl acetylacetonate.

The monophosphine is one or more of triphenylphosphine, tris(o-tolyl)phosphine, trinaphthylphosphine, tri(p-methoxyphenyl)phosphine, tri (m-chlorophenyl)-phosphine, tribenzylphosphine, tricyclohexylphosphine, dicyclohexylphenylphosphine, cyclohexyldiphenylphosphine, and trioctylphosphine. The monophosphine is triphenylphosphine in some embodiments. In some embodiments, the catalyst composition comprises a mixture of different species of monophosphines.

Once the step of contacting the C₁₂ linear olefin with carbon monoxide, hydrogen and a catalyst composition has produced the aldehyde mixture, next the step of hydrogenating the aldehyde mixture to produce the alcohol mixture comprising greater than 90 wt % of linear tridecanol based on the total weight of the alcohol mixture is performed. It will be understood that the mixture formed from the hydrogenation of the aldehyde mixture will contain residual olefins and aldehydes and that greater than 90 wt % of the alcohols produced (i.e., the alcohol mixture) will be linear tridecanols. As used herein, hydrogenation is the chemical reaction between molecular hydrogen (H₂) and an aldehyde which saturates the aldehyde into an alcohol. Hydrogenation is typically carried out in the presence of a catalyst such as nickel, palladium or platinum.

After the step of hydrogenation of the aldehyde mixture to form the alcohol mixture is performed, the alcohol mixture may be distilled to further increase the wt % of linear tridecanols present. Distillation is used to separate the linear tridecanol from one or more other materials it is mixed with (e.g., un-reacted olefins, solvents, aldehydes, etc.). The distillation is performed using a distillation column and may be performed at temperatures between 100° C. and 500° C.

### Method of Making the Surfactant Material

The present disclosure is also directed to the formation of a surfactant material. The surfactant material is formed from the alcohol mixture and is thus a derivative of the alcohol mixture. The method of making the surfactant material comprises the step of performing an alkoxylation of the alcohol mixture. Catalysts and reaction conditions for alkoxylation of alcohols are well known in the art as described in “Alkylene Oxides and Their Polymers” edited by F. E. Bailey, Jr. and Joseph V. Koleske, Marcel Dekker, Inc., New York, 1991. The alcohol mixture may be alkoxylated with ethylene oxide, propylene oxide, butylene oxide and combinations thereof. The alkoxylation of the alcohol mixture generates the surfactant material. Surfactant materials that can be derived from the alcohol mixture of this invention also include alkyl polyglucosides using a synthetic approach as described in “Nonionic Surfactants-Alkyl Polyglucosides”, edited by Dieter Balzer and Harald, Marcel Dekker, Inc., New York, 2000, pp 19-75, and alcohol and alcohol ether sulfate anionic surfactants as described in “Anionic Surfactants-Organic Chemistry” edited by Helmut W. Stache, Inc., New York, 1996, pp 223-312.

### EXAMPLES

### Materials

The following materials were used in the examples.

Olefin is a 12 carbon alpha-olefin commercially available as NEODENE™-12 from Shell Chemical LP, Houston, Texas.

Hydrogenation Catalyst is a nickel-based hydrogenation catalyst and is commercially available as Ni 3228 from BASF, Ludwigshafen, Germany.

Rhodium is rhodium dicarbonyl acetylacetonate and is available from MilliporeSigma, Burlington, MA.

TPP is triphenyl phosphine and is available from MilliporeSigma, Burlington, MA.

Solvent is toluene and is available from MilliporeSigma, Burlington, MA.

Tetraphosphine used in the examples and is prepared as set forth below.

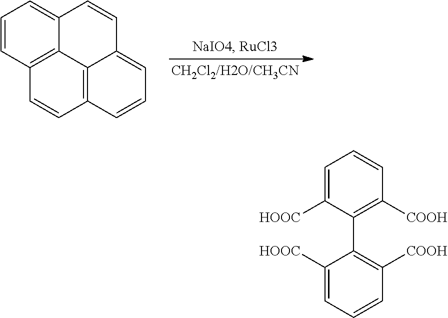

Synthesis of 1,1′-biphenyl-2-2′,6,6′-tetracarboxylic acid. A 5 L jacketed reactor equipped with an overhead stirrer, bottom drain valve and water cooled condenser is charged with 1 L of methylene chloride and 50 g (0.247 mol) of pyrene. The mixture is stirred until the pyrene dissolves, after which 0.25 L of acetonitrile, 1.5 L of deionized water and 2.0 g of ruthenium (III) chloride are added. The resulting biphasic mixture is stirred vigorously and cooled to 18° C. by circulating cooling fluid through the jacket. Sodium periodate is then added in small portions (500 g total; 2.34 mol) over a period of 2.5 hours while maintaining a reactor temperature of 23° C.-27° C. The reaction mixture which is initially brown in color quickly turns dark brown and finally brownish green. After stirring overnight (18 hours) agitation is stopped and the layers allowed to separate. The lower layer is drained into a Buchner funnel to collect the crude green/brown solid product which is washed with methylene chloride (2×500 mL) and dried on the filter by flowing air. The solid is then returned to the reactor and refluxed with 1.5 L of acetone for 1 hour. After cooling to 23° C., the yellow solution is drained into a Buchner funnel and the filtrate concentrated on a rotary evaporator leaving a yellow solid. The crude tetraacid product is dried in a vacuum oven at 70° C. overnight and used without further purification.

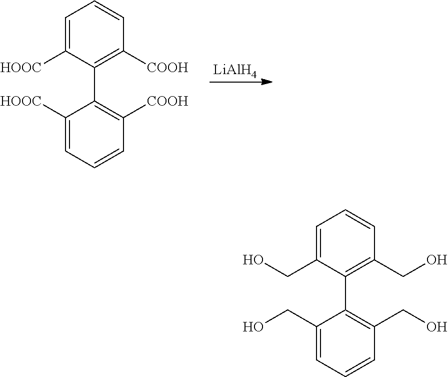 [SMILES: O=C(O)c1cccc(C(=O)O)c1-c1c(C(=O)O)cccc1C(=O)O ; "LiAlH4" ; OCc1cccc(CO)c1-c1c(CO)cccc1CO]

Synthesis of 1,1′-biphenyl-2,2′,6,6′-tetramethanol. The 5 L reactor used in the previous step is dried and purged with nitrogen overnight. Crude 1,1′-biphenyl-2-2′,6,6′-tetracarboxylic acid (50.0 g, 0.152 mol) is charged along with 1.5 L of THF under nitrogen. The resulting solution is stirred and cooled to 0° C. by circulating chilled fluid through the jacket of the reactor. A solution of lithium aluminum hydride in THF (1M; 666 mL; 0.665 mol) is then added via a peristaltic pump over 2 hours. During this time the mixture is stirred vigorously and the reactor temperature is maintained at 0° C. to 2° C.; for safety purposes a slow purge of nitrogen is applied to the reactor and the vent stream is passed through a condenser to sweep the reactor of evolved hydrogen. After the lithium aluminum hydride addition is complete, the reactor is stirred cold for an additional 15 minutes, then allowed to warm slowly to room temperature. After stirring at room temperature for 30 minutes, the reactor contents are heated to 65° C. and stirred overnight under a slow nitrogen purge. The next morning the reactor is cooled to 0° C. and quenched with 25 mL of water added slowly via the peristaltic pump, followed by 50 mL of 10% NaOH and 75 mL of water at 0° C. to 7° C. over a period of 1.5 hours. The quench procedure evolves hydrogen, and is therefore performed with a nitrogen sweep. The quenched solution is allowed to warm slowly to room temperature and then drained from the reactor into a Buchner funnel. The solids thus collected are washed with hot THF (3×300 mL). The volatiles are removed from the combined filtrate on a rotary evaporator to leave 35 g of a light yellow solid. The solids were dissolved in hot ethanol, filtered, and the solvent removed on a rotary evaporator.

Drying overnight in a vacuum oven left 32.3 g of light yellow product (77.1% yield, ca. 97% purity). ¾ NMR (400 MHZ, DMSO). d 7.46 (d, J=6.8 Hz, 4H), 7.39 (dd, J=8.6, 6.4 Hz, 2H), 4.99 (t, 7=5.3 Hz, 4H), 3.94 (d, 7=5.3 Hz, 8H) ppm. ¹³C NMR (400 MHZ, DMSO) d 139.3, 133.1, 127.3, 125.4, 60.4 ppm.

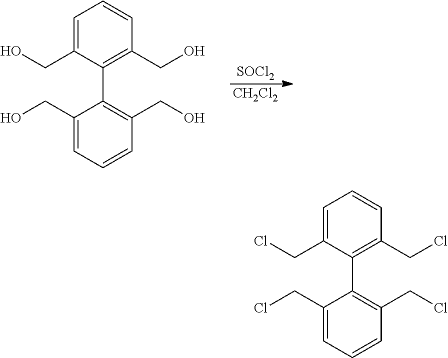

Synthesis of 2,2′,6,6′-tetrakis(chloromethyl)-1,1′-biphenyl. The 5 L reactor is dried and purged with nitrogen overnight and then charged with 1,1′-biphenyl-2,2′,6,6′-tetramethanol (45 g; 0.164 mol), methylene chloride (450 mL) and dimethylformamide (1 mL). The resulting yellow solution is stirred and cooled to 0° C. Thionyl chloride (1,071 g, 9.01 mol) is then added slowly via peristaltic pump over a 2 hour period, keeping the reactor temperature near 0° C.; during the addition the reactor is swept with nitrogen to remove the HCl and SO₂ which are generated, with the off gases passed through a water scrubber. The reaction solution is then allowed to warm to 23° C. and stirred for 30 minutes before heating to reflux (ca. 45° C.) overnight. The next day, the solution was cooled to 15° C. and discharged from the reactor. The methylene chloride was removed by distillation at atmospheric pressure, and the residual thionyl chloride removed by vacuum distillation. The resulting residue was dried first on a rotary evaporator followed by drying in a vacuum oven at 60° C. overnight to leave 58.1 g of yellow solid. (100% yield, ca. 95% purity). ¾ NMR (400 MHZ CDCl2) d 7.66-7.60 (m, 4H), 7.56 (dd, 7=8.8, 6.4 Hz, 2H), 4.28 (s, 8H) ppm. ¹³C NMR (400 MHZ, CDCl2) d 136.9, 135.5, 131.3, 130.3, 45.0 ppm.

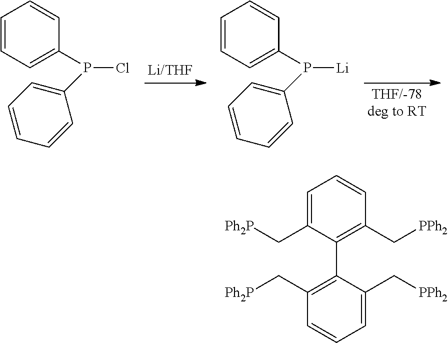

Synthesis of (1,1′-biphenyl-2,2′,6,6′-tetramethanediyl)tetrakis(diphenylphosphane) (Tetraphosphine). Lithium wire (2.1 g, 300 mmol) is cut into small pieces and charged into a 250 mL flask in a dry box along with anhydrous THF (130 mL). The suspended solution is transferred to a Schlenk line and chilled in an ice water bath under nitrogen. Chlorodiphenylphosphine (28.1 mL, 151.7 mmol) was added dropwise at 0° C. over a period of 50 minutes and then stirred an additional 30 minutes at 0° C. During this time the color changes from cloudy yellow to red. The solution was transferred to a dry box and stirred at room temperature overnight. The next morning the solution was cannula filtered into a clean, dry 500 mL round bottom flask, transferred to the Schlenk line and chilled to −78° C. A solution of 2,2′6,6′-tetrakis(chloromethyl)-1,1′-biphenyl (12.7 g, 37 mmol) in THF (60 mL) was added dropwise over 50 minutes, and then stirred cold for an additional 20 minutes. The solution is then allowed to warm slowly to 23° C., and then transferred to the dry box and stirred overnight. Degassed methylene chloride (300 mL) and water (150 mL) were then added, and the resulting mixture allowed to separate. The lower layer was transferred to a round bottom flask and concentrated on a rotary evaporator at 30° C. to leave a solution of crude product in THF. While heating this solution at 65° C. under flowing nitrogen, degassed ethanol (100 mL) is added slowly. White solid began precipitating during the ethanol addition. The mixture was then allowed to cool and placed in a refrigerator overnight; the resulting solids are collected the next day by filtering in the dry box, and washing with ethanol (2×50 mL). Drying under vacuum overnight leaves the desired product as a white powder (90% yield, 99% purity). ³¹P NMR (400 MHZ, CDCl3) d-14.5 ppm. ¾ NMR (400 MHZ, CDCl3) d 7.30-7.17 (m, 40H), 6.91-6.82 (m, 2H), 6.72 (d, J=7.7 Hz, 4H), 3.21 (s, 8H) ppm. The resulting tetraphosphine has structure (III).

### Test Method

Gas Chromatographic (“GC”) analyses are performed on an Agilent 6890 gas chromatograph using the parameters detailed in Table 1.

TABLE 1

| | |
|---|---|
| stationary phase | DB-1 |
| dimensions | 30 m × 0.32 mm |
| injection | split; 30:1 |
| inlet temp; ° C. | 250 |
| oven ramp | 40° C. hold 5 minutes; to |
| | 265° C. @ 20°/minute; hold |
| | 10 minutes |
| detector | FID |
| quantitation | Area % |

### Sample Preparation and Results: Aldehyde Mixture

The combined results of Examples 1-4 are summarized in Table 2.

Example 1: A 100 mL Parr mini reactor was charged with a solution comprising the Rhodium (0.0121 g; 150 parts per million (“ppm”) rhodium), Tetraphosphine (0.0886 g; 2 mol/mol rhodium), TPP (0.6538 g; 2 wt %), Olefin (30 mL; 23.6 g) and Solvent (10 mL; 8.7 g). The reactor was pressurized with 1:1 CO:H₂ (syngas; 0.206 megapascals (“MPa”)) and heated with stirring. When a temperature of 95° C. was reached, the pressure was increased to 0.689 MPa with 1:1 syngas. The reactor pressure was then maintained at 0.689 MPa throughout the run by controlled introduction of syngas using a Brooks mass flow meter and Brooks totalizer. After 4 hours at 95° C. the heat was turned off and the reactor was allowed to cool. The aldehyde mixture was removed from the reactor and analyzed by GC.

Example 2: The procedure of Example 1 was repeated, with the exception of the use of lower rhodium concentration, lower reaction temperature, lower reaction pressure and a longer reaction time.

Example 3: The procedure of Example 1 was repeated, with the exception of a higher concentration of TPP, a lower reaction temperature and pressure and a shorter reaction time.

Example 4: The procedure of Example 1 was repeated, with the exception of a higher concentration of TPP, a lower reaction temperature and pressure.

The % olefin conversion calculation of Table 2 is based on the unreacted olefin content of the aldehyde mixture. For example, a 90% olefin conversion indicates that the aldehyde mixture, exclusive of solvent content, is comprised of 10% unreacted olefins. The % linear aldehyde calculation of Table 2 is based on the relative amounts of aldehyde isomers in the aldehyde mixture. By way of illustration, the aldehydes contained in the aldehyde mixture of Example 4 are comprised of 96% linear aldehyde and 4% branched aldehydes. In Table 2, the abbreviation “Rx” stands for reaction.

TABLE 2

| | | Rh | | | | | |
|---|---|---|---|---|---|---|---|
| | Rx | concen- | | Rx | Rx | Olefin | |
| | time | tration | TPP | temp | pressure | conversion | % linear |
| Ex | (hr) | (ppm) | (wt %) | (° C.) | (MPa) | (%) | aldehyde |
| 1 | 4 | 150 | 2 | 95 | 0.689 | 88 | 96.6 |
| 2 | 5 | 100 | 2 | 90 | 0.414 | 90 | 96.3 |
| 3 | 2 | 100 | 4 | 90 | 0.414 | 89 | 95.3 |
| 4 | 4 | 100 | 4 | 80 | 0.414 | 78 | 96.0 |

The combined results of Examples 5-9 are summarized in Table 3.

Example 5: A 300 mL Parr mini-reactor was charged with a solution comprising the Rhodium (0.0377 g; 100 ppm rhodium), Tetraphosphine (0.2764 g; 2 mol/mol rhodium), TPP (0.6538 g; 0.4 wt. %), Olefin (170 mL; 133.5 g) and Solvent (20 mL; 17.3 g). The reactor was pressurized with 1:1 CO:H₂ (syngas; ca. 0.206 MPa) and heated with stirring. When the reactor temperature reached 90° C., the pressure was increased to 0.414 MPa with 1:1 syngas. The reactor pressure was then maintained at 0.414 MPa throughout the run by controlled introduction of syngas using a Brooks mass flow meter and Brooks totalizer. After 5 hours at 90° C. the heat was turned off and the reactor is allowed to cool. The aldehyde mixture was removed and analyzed by GC.

Examples 6-9: The procedure of Example 5 is repeated except for the use of 2 wt % TPP as well as slight variation in the concentrations of Olefin, Solvent, and reaction times.

TABLE 3

| | | Rh | | | | | |
|---|---|---|---|---|---|---|---|
| | Rx | Concen- | | | | Olefin | |
| | time | tration | TPP | Olefin | Solvent | conversion | % linear |
| Rx | (hr) | (ppm) | (wt %) | (g) | (g) | (%) | aldehyde |
| 5 | 5 | 100 | 0.4 | 133.5 | 17.3 | 77 | 96.9 |
| 6 | 7 | 150 | 2 | 116.2 | 14.3 | 67 | 95.2 |
| 7 | 7.5 | 150 | 2 | 121.7 | 16.0 | 78 | 93.4 |
| 8 | 6 | 100 | 2 | 145.3 | 7.4 | 74 | 94.6 |
| 9 | 7 | 150 | 2 | 145.2 | 10.4 | 69 | 93.0 |

### Sample Preparation and Results: Linear Tridecanol

A composite was prepared by combining the aldehyde mixtures of Examples 1-9. In a series of reactions, a portion (e.g., 200 mL) of the composite was hydrogenated in a 300 mL stainless steel Parr reactor to form an alcohol mixture. For the initial reaction, the Hydrogenation Catalyst was loaded into a catalyst basket, the reactor was then assembled, purged and leak checked with nitrogen. The reactor was slowly brought to reduction temperature (e.g., <150° C.) and hydrogen was fed with careful control to avoid temperature spikes. Once the reduction process was complete, the composite aldehyde mixture was vacuum transferred into the reactor. The reactor was then pressurized with hydrogen and brought to reaction temperature (140° C.). Hydrogen pressure was maintained at 3.89 MPa (absolute) throughout by means of a Brooks mass flow meter. Once the hydrogenation reaction was completed, the reactor was cooled, depressurized, and the alcohol mixture was collected through a drain valve in the reactor. A fresh portion of the composite was then vacuum transferred into the reactor and a second hydrogenation reaction was performed under the same conditions. In this fashion about 600 g of the composite aldehyde mixture was hydrogenated in a series of three reactions to provide approximately 600 g of an alcohol mixture.

The resulting alcohol mixture was distilled using a spinning band distillation column from B/R Instrument Corporation. During distillation, the alcohol mixture was loaded into a 1-L bottom distillation reboiler, connected to the bottom of the column, and placed into a heating mantle. A magnetic stir bar was used to achieve good mixing and even boiling. Fractions were collected based on boiling point such as is known in the art; a lights fraction comprised of alkanes and unreacted olefins is collected and discarded; the alcohol fractions are recombined and hydrogenated a second time. The resulting alcohol mixture (460 g) was distilled a second time. Various overhead cuts are taken during the distillation based on boiling point and/or the volume of distillate in the receiver; a small residue (bottoms) remains in the reboiler. Results of the second distillation are summarized in Table 4.

TABLE 4

| | | | | | | | | | Composite |
|---|---|---|---|---|---|---|---|---|---|
| Sample | Cut 1 | Cut 2 | Cut 3 | Cut 4 | Cut 5 | Cut 6 | Cut 7 | Bottoms | of cuts 2-7 |
| 1- | 82.6 | 92.9 | 94.8 | 95.8 | 96.6 | 98.0 | 99.1 | NA | 95.6 |
| tridecanol | | | | | | | | | |
| wt. % | | | | | | | | | |

The results of Table 4 show that an alcohol mixture comprising ≥90 wt % linear tridecanol may be prepared using the process of the present invention. The balance of the alcohol mixture is branched tridecanols.

### Sample Preparation and Results: Surfactant

A sample of the distilled alcohol mixture from above was alkoxylated to demonstrate the alcohol mixture's suitability as a precursor for manufacturing surfactants. The alcohol mixture (94.1 g) and 2.1 g of 45% aqueous KOH were charged to a round bottom flask and stripped by rotoevaporation at 75° C. to a final water content less than 500 ppm. The catalyzed alcohol (74.48 g) was charged to a 2000 mL Parr stainless steel reactor equipped with an impeller, a dip tube (¼″ inch OD) that is connected to nitrogen line and oxide feed line. The reactor was inerted by a nitrogen pad/de-pad sequence (6 cycles). The reactor was heated to 145° C. under 0.154 MPa (absolute) nitrogen pressure. Heating is provided by an external electric band heater and cooling was provided by water circulated through an internal coil with flow control. Ethylene oxide (67.98 g, 1.54 moles) was charged to the reactor while maintaining a pressure of less than 0.399 MPa (absolute) and digested for 1 hour upon charge completion to form an ethoxylated product. The ethoxylated product was cooled to 65° C. and drained from the reactor (141 g) to obtain a mass balance. The actual amount of ethylene oxide charged to the reactor by mass difference was 67.98 g to produce an ethoxylated product with a theoretical molecular weight of 378 g/mol. The ethoxylated product was neutralized with glacial acetic acid (0.803 g).

1.00 gram of the ethoxylate product was dissolved in 1000 ml of deionized water to make a solution at 0.1 wt % concentration. Surface tension of the solution was measured on KRÜSS K100 force tensiometer using the Wilhelmy plate method at room temperature. The surface tension was reported as 26.4 mN/m, indicating the ethoxylate product is a surfactant material.

What is claimed is:

1. An alcohol mixture, comprising:
greater than 90 wt % of a linear tridecanol based on the total weight of the alcohol mixture.

2. The alcohol mixture of claim 1, wherein 92 wt % or greater of the alcohol mixture is linear tridecanol based on the total weight of the alcohol mixture.

3. The alcohol mixture of claim 2, wherein 94 wt % or greater of the alcohol mixture is linear tridecanol based on the total weight of the alcohol mixture.

4. The alcohol mixture of claim 1, wherein 7 wt % or less of the alcohol mixture is branched tridecanol based on the total weight of the alcohol mixture.

5. A formulation, comprising:
0.1 wt % to 99 wt % of the alcohol mixture of claim 1.

6. A method of making an alcohol mixture, comprising the steps of:
contacting a C₁₂ linear olefin with carbon monoxide, hydrogen and a catalyst composition to produce an aldehyde mixture; and
hydrogenating the aldehyde mixture to produce an alcohol mixture comprising greater than 90 wt % of linear tridecanol based on the total weight of the alcohol mixture.

7. The method of claim 6, further comprising the step of:
distilling the alcohol mixture.

8. A method of making a surfactant material, comprising the step of:
performing an alkoxylation of the alcohol mixture of claim 6.
